(19) United States
(12) Patent Application Publication
(10) Pub. No.: US 2008/0146579 A1
(43) Pub. Date: Jun. 19, 2008

(54) TREATMENT OF PATIENTS WITH CHRONIC PULMONARY DISEASES AND NUTRITIONAL COMPOSITIONS THEREFORE

(75) Inventors: Erwin Peke MEIJER, Wageningen (NL); George Verlaan, Wageningen (NL); Maarteen Anne Hoijer, Arnhem (NL)

Correspondence Address: FOLEY AND LARDNER LLP;SUITE 500, 3000 K STREET NW, WASHINGTON, DC, 20007, US

(73) Assignee: N.V. Nutricia

(21) Appl. No.: 11/611,562

(22) Filed: Dec. 15, 2006

(51) Int. Cl. A61K 31/519; A23L 1/302; C07D 307/20; C07D 475/02; A61K 31/341

(52) U.S. Cl. 514/250; 426/72; 514/473; 544/257; 549/476

(57) ABSTRACT

The invention pertains to a method and compositions for treatment or prevention of chronic pulmonary dysfunction in a patient.

### FIELD OF THE INVENTION

This invention pertains to a method for treatment or prevention of chronic pulmonary dysfunction in a patient. The supplements are especially beneficial in stopping the progression of or reversing the symptoms of chronic pulmonary dysfunction.

### BACKGROUND OF THE INVENTION

Chronic pulmonary diseases are multifactor diseases often characterized by the presence of an increased energy depletion and an increase in oxidative stress both caused by the dysfunction of the lungs. Chronic pulmonary diseases are diseases such as emphysema, chronic asthma, chronic obstructive pulmonary disease (COPD), chronic bronchitis, pulmonary fibrosis, and pulmonary embolism. These chronic pulmonary diseases are a major health problem. COPD for example

is a leading yet under-recognized cause of morbidity and mortality worldwide (Chapman et al. Eur Respir J. 2006 January; 27(1):188-207). The prevalence of COPD in the general population is estimated to be ˜1% across all ages rising steeply to ˜10% amongst those aged >40 yrs. The prevalence climbs appreciably higher with age. Not surprisingly, the economic burden in Western countries is increasing. The economic burden of COPD in the UK, for example, has been estimated between £781 and £1,154 per patient per year with an overall annual cost (both direct and indirect) of between ±800M and £1,500M (Halpin and Miravitlles Proc Am Thorac Soc. 2006 September; 3(7):619-23.). Treatment of chronic pulmonary diseases commonly is, however, merely symptomatic.

WO 2006116353 discloses the use of a composition comprising N-acetyl cysteine and folate for the treatment of pulmonary diseases. This composition is supposed to have beneficial anti-oxidant effects.

WO 2005027935 discloses the use of several cysteine sources for the treatment of inflammatory pulmonary diseases.

WO 2004062656 discloses the use of glutamate and glutamate precursors for the treatment of COPD.

EP 0656754 discloses the use of a specific w-3 fat blend for the treatment of pulmonary patients.

The disadvantage of the known treatments is that they do not disclose an optimal nutritional treatment that is focused on the problems related to pulmonary function, i.e. increased energy depletion and an increase in oxidative stress. Thus the need remains for a nutritional therapy to limit the progression of chronic pulmonary diseases.

### SUMMARY OF THE INVENTION

The invention relates to a method for treatment or prevention of chronic pulmonary diseases in a patient, said method comprising administering to a patient with a chronic pulmonary disease a nutritional composition comprising effective amounts of D-ribose and N-acetyl cysteine (NAC).

For certain jurisdictions outside the US the present invention is worded as the use of D-ribose and N-acetyl cysteine in the manufacture of a nutritional composition for treatment or prevention of chronic pulmonary diseases or in an alternative wording a composition comprising D-ribose and N-acetyl cysteine for treatment or prevention of chronic pulmonary diseases.

The combination of D-ribose and NAC has beneficial effects on pulmonary function compared to D-ribose or NAC alone. In an advantageous embodiment in addition to administering the combination of D-ribose and N-acetyl cysteine further an effective amount of folate is administered. In one embodiment the further effective amount of folate that is administered is between 100 micrograms to 20 milligrams folate per day.

In one embodiment the effective amount of ribose is 0.5 to 40 grams D-ribose per day.

In one embodiment the effective amount of NAC is at least 100 mg per day or at least about 200 mg per day or at least about 400 mg per day or at least about 600 mg per day or at least about 1000 mg per day with a maximum of 10 g.

A further improvement of the pulmonary function can be obtained when additionally EPA, DHA and/or GLA is administered with the nutritional composition to a patient with a chronic pulmonary disease or alternatively to a patient with decreased lung function.

In one aspect the invention relates to a composition that is of benefit to patients with chronic pulmonary diseases, said composition comprising D-ribose and N-acetyl cysteine and at least one n-3 fatty acid selected from the group consisting of EPA, DHA, and GLA. In one embodiment the composition further comprises vitamin C and vitamin E. Another embodiment is a complete nutritional composition having a nutritional matrix comprising protein, fat and carbohydrates where D-ribose and N-acetyl cysteine and at least one n-3 fatty acid selected from the group consisting of EPA, DHA, and GLA are included in the nutritional composition.

### DETAILED DESCRIPTION OF THE INVENTION

Ingredients

Ribose: The term ribose is intended to include D-Ribose and other related compounds that are readily converted to ribose in vivo or which spare endogenous ribose. These compounds include ribitol, ribulose, 5-phosphoribose, xylitol, xylulose and sedoheptulose. Essentially pure, crystalline D-ribose is preferred. However, partially pure ribose isolated from yeast or produced by a bacterial fermentation may also be used, provided that the effective amount of D-ribose is contained in the crude product.

Ribose may easily and inexpensively be synthesized, or be isolated from yeast or other natural sources, but is most conveniently produced by bacterial fermentation. The mother liquor can be any carbohydrate source, but is conveniently corn syrup. Likewise, the fermentation syrup or partially purified product may be used, provided that the amount of D-ribose is ascertained to be within the effective amounts described in this application. While essentially pure ribose is most desirable for formulations meant for human consumption, it is especially affordable to use a partially purified ribose for other mammals.

The effective amount of ribose is 0.5 to 40 grams D-ribose per day. In one embodiment the effective amount of D-ribose is one to 20 grams per day. In another embodiment the effective amount of D-ribose is one to 15 grams per day. In a beneficial embodiment a regimen is applied wherein the daily dose is administered in two, three or four portions.

N-acetyl cysteine (NAC): In vivo studies demonstrated that NAC attenuates the endotoxin-induced rise in pulmonary artery pressure that occur e.g. during sepsis or other bacterial infections. NAC is readily commercially available. Furthermore, synthetic cysteine equivalents, e.g. derivatives of N-acetyl cysteine, including salts solvates etc. and/or diacetylcysteine can be used. It is understood that a daily dosage can be subdivided into 2, 3 or more dosage units which may be taken several times a day.

Folate: The term folate is intended to mean folic acid, folate esters and salts of folic acids, precursors and substitutes thereof. Substitutes include other one-carbon donors for biosynthesis, including, without limit, biotin. Biotin is known as a cofactor in carbon dioxide transfer to carboxylase enzymes and thus functions as a one-carbon donor. Folic acid (Vitamin B9) is a multifunctional vitamin that is a one-carbon donor essential for many cellular syntheses, most importantly in the synthesis of the heme moiety, essential for the maturation of erythrocytes. Since the function of folate is as a one-carbon donor for biosynthesis, it may be conjectured that other one-carbon donor may substitute for folate in the methods of this invention. One such substitute may be biotin. The effective amount of folate is 100 micrograms to 20 milligrams folate per day and a more preferred effective amount of folate is 250 micrograms to 15 milligrams per day and a most preferred effective amount of folate is 500 micrograms to five milligrams per day.

In one embodiment when D-ribose, N-acetyl cysteine (NAC) and folate are to be administered, these ingredients are preferably combined in one formulation.

Omega 3 fatty acids: In another embodiment the composition may further comprise one or more of the omega 3 fatty acids Eicosapentaenoic acid (EPA), Docosahexaenoic acid (DHA), the omega-3 fatty acids found in fish oil, and gamma-linolenic acid (GLA). Without being bound by theory the omega 3 fatty acids probably improve the pulmonary symptoms in chronic pulmonary patients by moderating the inflammatory mechanisms that occur during chronic pulmonary diseases. In addition, beneficial effects have been shown of poly-unsaturated fatty acid supplementation on the response to exercise training in patients with COPD. In particular it was shown that, in addition to the beneficial effects of pulmonary rehabilitation, functional capacity in patients with COPD was increased after 8 weeks of poly-unsaturated fatty acid supplementation compared with placebo as shown by improvements in peak exercise capacity and submaximal endurance time.

In one embodiment at least 1 gram per day of EPA and DHA and more preferably 2 grams per day are co-administered or included in the composition of the invention since trials using over 2 grams per day of omega-3 (as typically found in ten grams of fish oil) reported significant anti-inflammatory effects, beneficial for patients with chronic pulmonary diseases. In an advantageous embodiment EPA and DHA are present in combination with gamma-linolenic acid (GLA). This combination is of benefit because GLA partly replaces EPA and DHA with similar efficacy on blood pressure, thereby improving the taste of a composition comprising fish oil.

Additional benefit is obtained in a current embodiment when D-ribose, NAC and folate is combined with EPA and DHA and/or GLA to improve the energy status of patients with chronic pulmonary diseases such as emphysema, chronic asthma, chronic obstructive pulmonary disease (COPD), chronic bronchitis, pulmonary fibrosis, and pulmonary embolism. Skeletal muscle weakness is frequently observed in patients with COPD and plays a pivotal role in exercise intolerance. Furthermore, Steiner et al. (Thorax. 2005; 60(11):932-6) showed that the ATP demands of exercise were not met by resynthesis from oxidative and non-oxidative sources. These results suggested that significant metabolic stress occurs in the skeletal muscles of COPD patients during whole body exercise at low absolute workloads similar to those required for activities of daily living.

Betaine: Betaine significantly improves the glycogen energy storage in muscles and thereby improves the energy status of the patients. Further improvement of the method and the composition according to the invention can be obtained when betaine (trimethyl glycine) is included in the composition and is also administered to a patient with chronic pulmonary disease.

Coenzyme Q10 (CoQ10) supplementation has been shown to significantly increase the energy status of a person. At higher doses than 30 mg, the dose dependent increase of plasma CoQ10 is found to be linear at least up to a daily dose of 200 mg, which results in a more than six-fold increase in plasma CoQ10 levels. Coenzyme Q10 enhances the efficient use of the increased energy storage thereby supporting the effect of betaine and remitting the wasting of muscle tissue. Preferably both CoQ10 and betaine are included in the composition according to the invention and are administered in the method according to the invention.

An effective amount is at least 50 mg of CoQ10 per day. In one embodiment the amount of CoQ10 is 100 to 500 mg CoQ10 per day. An effective amount of betaine is between 50 and 50000 mg per day, in other embodiments between 100 and 10000 mg per day and between 1000 and 10000 mg per day.

Interestingly, the inventors showed using in vitro experiments in liver cells that incubation with increasing concentrations of folic acid was able to restore the nucleotide status. Moreover, addition of folic acid in combination with D-ribose and NAC showed a synergetic increase in the adenosine nucleotide pool and total nucleotide pool. Thus, the combination of folic acid with D-ribose and NAC is more effective to improve cellular energy status than ribose, NAC or folic acid alone.

Without being bound by theory, the mechanism by which NAC improves the exercise capacity is believed to work through reducing the local oxidative stress produced by the mitochondrial electron transport chain which affects the energy status of the skeletal muscle. In addition, it has been clearly shown that significant metabolic stress occurs (e.g. increased ATP demands) in the skeletal muscles of COPD patients during whole body exercise at low absolute workloads similar to those required for activities of daily living.

Vitamin E and C: The effects of oxidative stress on muscle metabolism could be further reduced by using an effective amount of vitamin E and C. Therefore, in an embodiment the invention comprises the administration of effective amounts of D-ribose and NAC in combination with folate, and at least one n-3 fatty acid selected from the group consisting of EPA, DHA and GLA, and vitamin E and vitamin C.

N-acetyl cysteine, Betaine, Coenzyme Q10, EPA, DHA, and GLA, and vitamin E and C are thus agents that can beneficially be used to improve the energy status of a pulmonary patient.

A further embodiment of the invention is a composition comprising per 100 gram dry weight 5-20 g D-ribose and 0.3-10 g N-acetyl cysteine and 0.1 to 5 g of at least one n-3 fatty acid selected from the group consisting of EPA, DHA, and GLA. The composition may further comprise per 100 gram dry weight 0.05-10 microgram folate.

These ranges are specifically suitable for use in a clinical nutritional composition according to the applicable regulations. Additionally the composition may further comprise vitamin E and vitamin C, preferably in the range according to the food regulations for medical purposes (FSMP). Additionally, the composition may further comprise betaine and/or coenzyme Q10.

A further embodiment of the invention is a complete nutritional composition that comprises apart from the ingredients described above also the more common major ingredients protein, fat and carbohydrate. Patients with pulmonary diseases are often have a deficit in the common nutritional ingredients and it is therefore much more comfortable and consequently improves compliance to administer the patients with such complete nutritional composition. The composition must be carefully chosen and has a preferred composition comprising:
D-ribose
N-acetyl cysteine
an amino acid source that provides at least 10% of the total caloric value of said food composition; and
fats that provide between 20 and 50% of the total caloric value of said food composition;
carbohydrates that provide the balance of the total caloric value of said food composition; and
at least one agent or 2, 3, 4, 5, 6, 7 or 8 agents selected from the group consisting of folate, betaine, coenzyme Q10, EPA, DHA, GLA, vitamin E and vitamin C.

Clinical Uses

Chronic obstructive pulmonary disease (COPD) that is recognized by both the American Thoracic Society and the European Respiratory Society is a preventable and treatable disease state characterized by airflow limitation that is not fully reversible. The airflow limitation is usually progressive and is associated with an abnormal inflammatory response of the lungs to noxious particles or gases, primarily caused by cigarette smoking. Although COPD affects the lungs, it also produces significant systemic consequences.

Chronic bronchitis is defined clinically as chronic productive cough for 3 months in each of 2 successive years in a patient in whom other causes of productive chronic cough have been excluded.

Emphysema is defined pathologically as the presence of permanent enlargement of the airspaces distal to the terminal bronchioles, accompanied by destruction of their walls and without obvious fibrosis.

Asthma differs from COPD in its pathogenic and therapeutic response, and should therefore be considered a different clinical entity. However, some patients with asthma develop poorly reversible airflow limitation. These patients are indistinguishable from patients with COPD but for practical purposes are treated as asthma.

The high prevalence of asthma and COPD in the general population results in the co-existence of both disease entities in many individuals. This is characterized by significant airflow limitation and a large response to bronchodilators. In these patients, the forced expiratory volume in one second (FEV1) does not return to normal and frequently worsens over time. There is a great need to design studies aimed at determining the prevalence, natural history, clinical course and therapeutic response in these patients.

### EXAMPLES

### Example 1

### Powder Composition According to the Invention

| Ingredient | g/100 g powder |
|---|---|
| D-ribose | 13 |
| Folic acid | 0.0001 |
| concentrated natural fish oil | 8 |
| Borage oil | 4 |
| fructose | 12 |
| Ca-caseinate | 6.5 |
| Na-caseinate | 6.5 |
| NAC (N-acetyl-L-cysteine) | 4 |
| Lecithin powder | 0.16 |
| Vit B2 | 0.0012 |
| Ascorbylpalmitate | 0.015 |
| carotenoid mix | 1.3 |
| trace element mix | 0.094 |
| Vitamin premix | 0.23 |

### Example 2

### Bar Composition According to the Intervention

| | Per |
|---|---|
| Ingredient | serving |
| D-ribose, g | 5.0 |
| Folic acid, mcg | 67 |
| Vitamin B12, mcg | 0.63 |
| EPA + DHA, g | 0.44 |
| GLA, g | 0.25 |
| Whey protein, g | 10 |
| Trehalose, g | 5.0 |
| NAC (N-acetyl-L-cysteine), mg | 200 |
| Vitamin C, mg | 167 |
| Vitamin E, mg α-tocopherol | 13.3 |
| equivalents | |
| Carotenoid mixture, mg | 1.0 |
| Galacto-oligosaccharides, g | 2.0 |

### Example 3

### A Pudding Composition According to the Intervention

A pudding for enteral administration to subjects with chronic pulmonary disease comprising 5 g D-ribose, 200 mg NAC, 67 mg folic acid, 20 IU vitamin E and 167 mg vitamin C. The pudding further comprises 20 En % whey protein, 60 En % carbohydrate and 20 En % lipid fraction consisting of EPA and DHA. En % is short for energy percentage and is the caloric contribution of the ingredient based on the total amount of calories of the composition. The pudding should be consumed 2-3 times a day.

1. A method for treatment or prevention of chronic pulmonary diseases in a patient, said method comprising administering daily to a patient with a chronic pulmonary disease a nutritional composition comprising effective amounts of D-ribose and N-acetyl cysteine (NAC).

2. The method according to claim 1 wherein the effective amount of ribose is 0.5 to 40 grams D-ribose per day.

3. The method according to claim 1 wherein the effective amount of NAC is at least 100 mg per day and at most 10 g per day.

4. The method according to claim 1 wherein the composition further comprises an effective amount of folate.

5. The method according to claim 4 wherein the effective amount of folate is between 100 micrograms to 20 milligrams per day.

6. The method according to claim 5 wherein the effective amount of D-ribose is 1 to 20 grams per day, the effective amount of NAC is at least 100 mg and at most 10 g per day and the effective amount of folate is 250 micrograms to 15 milligrams per day.

7. The method according to claim 5 wherein the effective amount of D-ribose is 1 to 15 grams per day, the effective amount of NAC is at least 100 mg and at most 10 g per day and the effective amount of folate is 500 micrograms to five milligrams per day.

8. A composition comprising per 100 gram dry weight 5-20 g D-ribose, 0.3-10 g N-acetyl cysteine and 0.1 to 5 g of at least one n-3 fatty acid selected from the group consisting of EPA, DHA, and GLA.

9. The composition according to claim 8 further comprising per 100 gram dry weight 0.05-10 microgram folate.

10. The composition according to claim 8 further comprising vitamin E and vitamin C.

11. The composition according to claim 9 further comprising vitamin E and vitamin C.

12. The composition according to claim 8 further comprising betaine and/or coenzyme Q10.

13. The composition according to claim 9 further comprising betaine and/or coenzyme Q10.

14. The composition according to claim 10 further comprising betaine and/or coenzyme Q10.

15. The composition according to claim 11 further comprising betaine and/or coenzyme Q10.

16. A nutritionally balanced food composition comprising
a. D-ribose;
b. N-acetyl cysteine;
c. an amino acid source that provides at least 10% of the total caloric value of said food composition;
d. fat that provides between 20 and 50% of the total caloric value of said food composition;
e. carbohydrate that provide the balance of the total caloric value of said food composition; and
f. one or more agents selected from the group consisting of folate, betaine, coenzyme Q10, EPA, DHA, GLA, vitamin C and vitamin E.
